(19) United States
(12) Patent Application Publication
(10) Pub. No.: US 2023/0366875 A1
(43) Pub. Date: Nov. 16, 2023

(54) GPCR19-P2XN RECEPTOR COMPLEX AND USE THEREOF

(71) Applicants: SEOUL NATIONAL UNIVERSITY R&DB FOUNDATION, Seoul (KR); SHAPERON INC., Seoul (KR)

(72) Inventor: Seung Yong SEONG, Seoul (KR)

(21) Appl. No.: 18/027,027

(22) PCT Filed: Aug. 23, 2021
(86) PCT No.: PCT/KR2021/011232

§ 371 Date: Mar. 17, 2023

Related U.S. Application Data
(60) Provisional application No. 63/080,127, filed on Sep. 18, 2020.

(51) Int. Cl. G01N 33/50; G01N 33/68

(52) U.S. Cl. CPC G01N 33/5041; G01N 33/6845; G01N 33/6872; G01N 2333/705

(57) ABSTRACT

The present invention relates to a GPCR19-P2Xn receptor complex and use of the same, particularly to a method for screening a substance that regulates the interaction between GPCR19 and a P2Xn receptor in their complex; a method for screening a substance for prevention or treatment of an NLRP3 inflammasome-associated diseases utilizing the interaction between GPCR19 and a P2Xn receptor in their complex; and a method for preventing or treating an NLRP3 inflammasome-associated disease, which comprises administering to an individual a pharmaceutically effective amount of a substance that induces the interaction between GPCR19 and a P2Xn receptor in their complex.

### TECHNICAL FIELD

The present invention relates to a GPCR19-P2Xn receptor complex and use of the same, particularly to a method for screening a substance that regulates the interaction between GPCR19 and a P2Xn receptor in their complex; a method for screening a substance for prevention or treatment of an NLRP3 inflammasome-associated diseases utilizing the interaction between GPCR19 and a P2Xn receptor in their complex; and a method for preventing or treating an NLRP3 inflammasome-associated disease, which comprises administering to an individual a pharmaceutically effective amount of a substance that induces the interaction between GPCR19 and a P2Xn receptor in their complex.

### BACKGROUND ART

Inflammasomes are cytosolic multiprotein oligomers of the innate immune system responsible for the activation of inflammatory response, and activation of the inflammasome promotes proteolytic cleavage of pro-inflammatory cytokines pro4L-1β and pro4L-18 into cytokines interleukin IL-1β and interleukin IL-18 and maturation and secretion thereof, as well as cleavage of gasdermin D through caspase-1. When dysregulation of such inflammasomal activation occurs, various diseases such as cancer, metabolic diseases, neurological diseases, degenerative diseases, and inflammatory diseases are caused.

For example, abnormal activation of NLRP3 inflammasome is associated with the onset and exacerbation of various inflammatory diseases such as ulcerative colitis, gout, multiple sclerosis, arthritis, sepsis, and inflammatory neurological disease. The NLRP3 inflammasome is activated by PAMP and calcium influx of pathogens such as viruses and bacteria, mitochondrial ROS, and DAMP stimulation such as extracellular ATP.

The P2X7 receptor is a major factor that exists in the cell membrane and regulates intracellular calcium ions with an ion channel, and is one of seven subtypes of the P2Xn receptors that act as a DAMP sensor such as the ATP. The P2X7 receptor plays a role in regulating the NLRP3 inflammasomal activation in high-level signaling steps.

As it is known that dysregulation of inflammasomes causes various diseases, the development of drugs targeting proteins involved in the inflammasome-related signals has been attracting attention since 2010. Drugs currently under development may be broadly divided into three categories of P2X7 receptor antagonists, NLRP3 inhibitors, and caspase-1 inhibitors.

Looking at the development status of each of the therapeutic agents, global pharmaceutical companies such as Pfizer, AstraZeneca, and Janssen started developing P2X7 receptor antagonists for indications such as rheumatoid arthritis, Crohn's disease, and depression, but most of them discontinued the development due to lack of efficacy in clinical trials. Since then, venture companies have continued to develop P2X7 receptor antagonists, but as of 2020, there are fewer than 10 drug development pipelines targeting P2X7. The new drug which is currently in the clinical stage is 18F-JNJ-64413739, which is being developed by Janssen as a therapeutic agent for depression and has passed through phase 1 clinical trials, and other pipelines are still in preclinical or development stages.

Caspase-1 inhibitors do not have a development pipeline as the development thereof was discontinued in 2020 due to lack of efficacy and safety issues in clinical trials.

NLRP3 inhibitors are being developed by venture companies such as OLATEC, INFLAZOME, IFM THERAPEUTICS, NODTHERA, and AC Immune for indications such as osteoarthritis, systolic heart failure, Parkinson's disease, inflammatory bowel disease, and Alzheimer's disease.

As described above, drugs targeting inflammasome-related signals are still in the initial development stage as less than 20 companies are attempting to develop the drugs worldwide as of 2020. As of 2020, there are three new drug pipelines that are undergoing clinical trials for the purpose of treating inflammatory diseases, and there are five new drug pipelines that are being developed in the preclinical stage. However, new drug candidates known to date have limitations in anti-inflammatory efficacy that selectively inhibit inflammatory cytokines triggered by inflammatory activity, such as IL-1β and IL-18, but cannot inhibit TNFα inflammatory cytokines triggered at the stage of initiation of inflammation. This is a basic limitation of new drugs targeting NLRP3 and P2X7, and it is due to the fact that drugs having a pharmacological mechanism that selectively blocks only the inflammatory activation phase cannot inhibit the inflammatory response through various inflammatory bypass pathways. There is also a disadvantage that the actual clinical efficacy response rate is low due to the redundancy and genetic polymorphism inherent in NLRP3 and P2X7.

Meanwhile, four types of GPCR-gated ion channels, GIRK1/2/3/4, are known so far, and GPCR-gated ion channels are known to be involved in physiological phenomena such as cardiomyocyte ion regulation, nerve pain signals, and alcoholism, but the exact mechanism and the relationship between the correlated receptors are not known in detail.

Accordingly, the present inventors have studied a pharmacological mechanism targeting the GPCR-gated ion channels as a method to overcome the limitations of drugs targeting inflammasome-related signals, as a result, newly revealed that P2X7 is a GPCR19-regulated ion channel, and thus achieved the present application. Specifically, the present inventors have newly revealed that a specific GPCR19 agonist regulates the P2X7 ion channel, which plays an important role in NLRP3 inflammasomal activation. In other words, a mechanism has been revealed in which GPCR19 up-regulates the P2X7 ion channel through mutual binding with the P2X7 receptor and the NLRP3 inflammasome is thus regulated. In addition, by revealing a pharmacological mechanism in which a substance that regulates the interaction between GPCR19 and P2X7 receptor in their complex inhibits inflammatory initiation-derived TNFα through GPCR19-CAMP signaling activity and at the same time inhibit inflammatory activity-derived IL-1β through GPCR19-mediated P2X7 signaling inhibition, the present application has been achieved.

### SUMMARY OF INVENTION

### Technical Problem

An object of the present invention is to provide a method for screening a substance that regulates the interaction between GPCR19 and a P2Xn receptor in their complex.

Another object of the present invention is to provide a method for screening a substance for prevention or treatment of an NLRP3 inflammasome-associated disease utilizing the interaction between GPCR19 and a P2Xn receptor in their complex.

Still another object of the present invention is to provide a method for preventing or treating an NLRP3 inflammasome-associated disease, which comprises administering to an individual a pharmaceutically effective amount of a substance that induces the interaction between GPCR19 and a P2Xn receptor in their complex.

### Solution to Problem

In order to achieve the objects, the present invention provides a method for screening a substance that regulates the interaction between GPCR19 and a P2Xn receptor in their complex, which comprises:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance having a difference in interaction between GPCR19 and a P2Xn receptor in their complex compared to a control group not treated with the candidate substance as a substance that regulates interaction between GPCR19 and a P2Xn receptor in their complex.

The present invention also provides a method for screening a substance for prevention or treatment of an NLRP3 inflammasome-associated disease, which comprises:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance that increases interaction between GPCR19 and a P2Xn receptor in their complex compared to a control group not treated with the candidate substance as a substance for prevention or treatment of an NLRP3 inflammasome-associated disease.

In addition, the present invention also provides a method for preventing or treating an NLRP3 inflammasome-associated disease, which comprises administering to an individual a pharmaceutically effective amount of a substance that induces the interaction between GPCR19 and a P2Xn receptor in their complex.

### Advantageous Effects of Invention

In the present invention, it has been confirmed that GPCR19 and P2X7 receptor bind and interact with each other. In addition, the physiological mechanism has been confirmed in which the NLRP3 inflammasomal activation pathway mediated by P2X7 is regulated by GPCR19 in the DAMP stress inflammation-induced situation due to biomaterials such as PAMP and ATP caused by microorganisms. In addition, it has been confirmed that the inflammatory response initiated from P2X7 can be prevented or alleviated when mutual binding between GPCR19 and P2X7 receptor is induced during an inflammatory response by utilizing a substance that induces mutual binding between GPCR19 and P2X7 receptor. Accordingly, it is possible to develop a preparation for preventing or treating NLRP3 inflammasome-associated diseases including inflammatory diseases by screening a substance that regulates the interaction between GPCR19 and P2X7 receptor and utilizing the screened substance.

### BRIEF DESCRIPTION OF DRAWINGS

FIG. 1A is a diagram confirming colocalization of GPCR19 and P2X7 receptor in keratinocytes by treatment with DNCB (2,4-dinitrochlorobenzene)+TNF-α±sodium taurodeoxycholate (hereinafter, referred to as ‘HY209’);

FIG. 1B is a diagram confirming colocalization of GPCR19 and P2X7 receptor in microglia by treatment with amyloid-β (Aβ)±ATP±HY209;

FIG. 1C is a diagram confirming colocalization of GPCR19 and P2X7 receptor in macrophages by treatment with LPS+BzATP±HY209;

FIG. 2A is a diagram confirming Ca⁺⁺ mobilization by P2X7 receptor after treatment of macrophages of a GPCR19 knockout mouse or a P2X7 knockout mouse with ATP;

FIG. 2B is a diagram confirming Ca⁺⁺ mobilization by P2X7 receptor after treatment of microglia of a GPCR19 knockout mouse or a P2X7 knockout mouse with ATP;

FIG. 2C is a diagram confirming Ca⁺⁺ mobilization by P2X7 receptor in keratinocytes by treatment with IL-1β/TNF-α+ATP±HY209;

FIG. 2D is a diagram confirming Ca⁺⁺ mobilization by P2X7 receptor in macrophages by treatment with LPS+ATP±HY209 or LPS+BzATP±HY209;

FIG. 2E is a diagram confirming Ca⁺⁺ mobilization by P2X7 receptor in microglia by treatment with Aβ+ATP±HY209 or Aβ+BzATP±HY209;

FIG. 3A is a diagram confirming changes in inflammasomal components in keratinocytes by treatment with DNCB+TNF-α±HY209;

FIG. 3B is a diagram confirming changes in IL-1β expression in keratinocytes by treatment with DNCB±TNF-α+ATP±HY209;

FIG. 4A is a diagram confirming changes in IL-1β expression in macrophages by treatment with LPS+ATP±HY209 or LPS+BzATP±HY209;

FIG. 4B is a diagram confirming changes in IL-1β expression by treatment with LPS±BzATP treatment and pre/post treatment with HY209;

FIG. 4C is a diagram confirming changes in inflammasomal components in macrophages by treatment with LPS+BzATP+HY209;

FIG. 5 is a diagram comparing the inflammatory response alleviating effect of a substance that induces interaction between GPCR19 and P2X7 receptor with that of a known inflammasome inhibitor;

FIG. 6 is a diagram confirming changes in P2X7 receptor, an ion channel by HY209, which is a GPCR19 agonist, through a potassium ion channel assay; and

FIG. 7 is a diagram schematically illustrating a mechanism according to the interaction between GPCR19 and P2X7 receptor.

### DESCRIPTION OF EMBODIMENTS

The present invention relates to a GPCR19-P2Xn receptor complex and use of the same, particularly to a method for screening a substance that regulates the interaction between GPCR19 and a P2Xn receptor in their complex; a method for screening a substance for prevention or treatment of an NLRP3 inflammasome-associated diseases utilizing the interaction between GPCR19 and a P2Xn receptor in their complex; and a method for preventing or treating an NLRP3 inflammasome-associated disease, which comprises administering to an individual a pharmaceutically effective amount of a substance that induces the interaction between GPCR19 and a P2Xn receptor in their complex.

### EMBODIMENTS

Hereinafter, the present invention will be described in detail by the following examples.

The present invention provides a vector comprising a gene encoding GPCR19 (G-protein coupled receptor 19) and a P2Xn receptor.

The present invention also provides a cell transformed with the vector.

The present invention also provides a GPCR19-P2Xn receptor complex isolated from the cell.

In the present invention, GPCR19 is a protein encoded by the human GPBAR1 gene, and is a member of the G-protein coupled receptor superfamily. GPCR19 functions as a cell surface receptor for bile acids. Specifically, when GPCR19 is activated by bile acids, cAMP is produced, the MAP kinase signal pathway is activated by cAMP, and the NF-κB action is in turn regulated.

In the present invention, P2Xn receptors are a member of the 2-transmembrane family. P2Xn receptors may play a role in rapid synaptic transmission, including non-selective cation channels. As P2Xn receptors, seven subtypes, more specifically P2X1, P2X2, P2X3, P2X4, P2X5, P2X6, and P2X7 receptors are known. In the present invention, P2X7 is preferred, but P2Xn receptors are not limited thereto.

In the present invention, the genes encoding GPCR19 and P2Xn may be used in the form of full length and/or fragments. Specifically, the genes include genes in which a part of the nucleotide sequence is artificially modified to favor features such as expression in cells or protein stability, genes in which a part of a naturally occurring nucleotide sequence is modified, or fragments of these as well as wild-type gene sequences encoding the proteins disclosed in the present invention and fragments thereof. The modification of a gene sequence may or may not involve modification of the corresponding amino acid. In the case of involving modification of amino acids, the gene in which this modification is induced is one that encodes a protein consisting of an amino acid sequence in which one or more amino acids are substituted, deleted, added and/or inserted in the protein encoded thereby, and includes mutants, derivatives, alleles, variants and homologues. When the mutation of a gene sequence does not involve the modification of amino acids in a protein, for example, there is a degenerate mutation, and such degeneracy mutants are also included in the gene of the present invention.

The artificial modification of a gene sequence may be performed by methods well known to those skilled in the art, for example, site-directed mutagenesis (Kramer et al, 1987), error-prone PCR (Cadwell, R. C. and G. F. Joyce. 1992. PCR methods Appl., 2:28-33.), and point mutation method (Sambrook and Russel, Molecular Cloning: A Laboratory Manual, 3rd Ed. 2001, Cold Spring Harbor Laboratory Press.).

In the present invention, the vector refers to a means for expressing a target gene in a host cell. The vector includes elements for the expression of the target gene, and may include a replication origin, a promoter, an operator, a terminator and the like, and may further include an appropriate enzyme site (for example, restriction enzyme site) for introduction into the genome of the host cell and/or a selectable marker to confirm successful introduction into the host cell and/or a ribosome binding site (RBS) for translation into a protein, IRES (internal ribosome entry site) and the like. The vector may further include a transcriptional regulatory sequence (for example, enhancer) other than the promoter.

The vector may be a plasmid DNA, a recombinant vector or another medium known in the art, and may specifically be a linear DNA, plasmid DNA, a recombinant non-viral vector, a recombinant viral vector or an inducible gene expression vector system, and the recombinant viral vector may be a retrovirus, an adenovirus, an adeno-associated virus, a helper-dependent adenovirus, a herpes simplex virus, a lentiviral vector, or a vaccinia virus, but the vector is not limited thereto.

In the present invention, the term “transformation” means that the genetic properties of an organism are changed by DNA given from the outside, that is, means a phenomenon in which when DNA, which is a type of nucleic acid extracted from a cell of a certain lineage of an organism, is introduced into a living cell of another lineage, the DNA enters the cell and the genetic trait is changed.

In the present invention, the gene encoding GPCR19 and a P2Xn receptor can be introduced into cells after a primer that can specifically recognize the gene from a known sequence as described above is prepared, the gene is amplified through the polymerase chain reaction using this primer, and this gene is introduced into the expression vector as described above. The method of introduction is known and includes, but is not limited to, for example, liposome mediated transfection, calcium phosphate method, DEAE-dextran mediated transfection, positively charged lipid mediated transfection, electroporation, transduction using a phage system or an infection using a virus.

The present invention also provides a method for screening a substance that regulates the interaction between GPCR19 and a P2Xn receptor in their complex, which comprises:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance having a difference in interaction between GPCR19 and a P2Xn receptor in their complex compared to a control group not treated with the candidate substance as a substance that regulates interaction between GPCR19 and a P2Xn receptor in their complex.

In the method according to the present invention, the candidate substance may be those presumed to have the potential to regulate the interaction between GPCR19 and a P2Xn receptor in their complex according to a conventional selection method, or may be randomly selected individual peptides, aptamers, antibodies, proteins, non-peptidic compounds, active compounds, fermentation products, cell extracts, plant extracts or animal tissue extracts, but is not limited thereto.

In the method according to the present invention, the first substance may be an inflammatory inducer or an NLRP3 inflammasome activator.

The inflammatory inducer may be a TLR (Toll-like receptor) ligand or a cytokine, for example, may be LPS (lipopolysaccharide), peptidoglycan, TNF-α, IL-1β or IL-17, but is not limited thereto, and any one may be used without limitation as long as it induces inflammation through the NF-κB signal transduction pathway.

The NLRP3 inflammasome activator may be amyloid-β (Aβ), but is not limited thereto.

In the method according to the present invention, the second substance may be a P2Xn receptor agonist, for example, may be ATP, BzATP, or nigericin, but is not limited thereto.

In the method according to the present invention, the P2Xn receptor may be P2X1, P2X2, P2X3, P2X4, P2X5, P2X6 or P2X7 receptor, and may specifically be P2X7 receptor, but is not limited thereto.

In the method according to the present invention, the cell may be stem cells, animal cells, insect cells, or plant cells, but is not limited thereto.

The stem cells may be specifically embryonic stem cells, adult stem cells, induced pluripotent stem cells (iPS), more specifically adult stem cells (mesenchymal stem cells), but are not limited thereto. The adult stem cells may be derived from various adult cells such as bone marrow, blood, brain, skin, fat, skeletal muscle, umbilical cord, and umbilical cord blood. Specific examples thereof include mesenchymal stem cells (MSC), skeletal muscle stem cells, hematopoietic stem cells, neural stem cells, hepatic stem cells, adipose-derived stem cells, adipose-derived progenitor cells, and vascular endothelial progenitor cells, but are not limited thereto.

The animal cells are a functional and structural basic unit originating from animals including humans, and cells originating from animals including humans (for example, mammals such as monkeys, dogs, goats, pigs, or cattle) may be included in the scope of the present invention. Accordingly, the animal cells of the present invention include, but are not limited to, myeloid cells, lymphoid cells, microglia, macrophages, more specifically bone marrow-derived macrophages, neutrophils, monocytes, epithelial cells, dermal cells, endothelial cells, muscle cells, germ cells, skin cells (for example, fibroblasts, keratinocytes), immune cells, cancer cells, and the like. Specific examples thereof include HaCat cells (human keratinocyte), BV2 cells (mouse microglia) CHO (Chinese hamster ovary) cells, NS0 (mouse myeloma) cells, BHK (baby hamster kidney) cells, Sp2/0 (mouse myeloma) cells, human retinal cells, HUVEC cells, HMVEC cells, COS-1 cells, COS-7 cells, HeLa cells, HEK-293 cells, HepG-2 cells, HL-60 cells, IM-9 cells, Jurkat cells, MCF-7 cells or T98G cells, but are not limited thereto.

The cell may heterologously or endogenously express GPCR19 and a P2Xn receptor, and may be introduced into cells as described above for heterologous expression.

In the method according to the present invention, the interaction between GPCR19 and a P2Xn receptor in their complex in step 3) may be measured by analyzing any one or more of the following characteristics:
i) a change in colocalization of GPCR19 and a P2Xn receptor in a cell surface, cytoplasm or nucleus;
ii) a change in GPCR19-mediated signal transduction pathway activity;
iii) a change in P2Xn receptor-mediated signal transduction pathway activity; and
iv) a change in inflammatory cytokine level.

Specifically, the change in GPCR19-mediated signal transduction pathway activity may be a change in cAMP level or PKA activity, but is not limited thereto.

The change in P2Xn receptor-mediated signal transduction pathway activity may be a change in Ca⁺⁺ mobilization or inflammasomal activation, and the change in inflammasomal activation may be a change in NLRP3, ASC, pro-IL-1β, IL-1β, pro-IL-18, IL-18, pro-caspase-1, caspase-1 or gasdermin D level, a change in NLRP3 inflammasome oligomerization, or a change in maturation of an IL-1β, IL-18 or caspase-1 immature form to a mature form, but the change is not limited thereto.

The change in inflammatory cytokine level may be a change in TNF-α, IL-1β, IL-18, RANTES or MCP-1 level, but is not limited thereto.

In the method according to the present invention, the characteristics may be measured by, for example, calcium ion assay, immunofluorescence method, immunoprecipitation method, protein chip analysis, western blotting, enzyme immunoassay (ELISA), RT-PCR (reverse transcription polymerase chain reaction), real-time RT-PCR, northern blotting, DNA chip analysis, ligand binding assay, radioimmunoassay, tissue immunostaining, or immunoassay, but is not limited thereto, and methods known in the art for analyzing the characteristics may be used without limitation.

The present invention also provides a method for screening a substance for prevention or treatment of an NLRP3 inflammasome-associated disease, which comprises:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance that increases interaction between GPCR19 and a P2Xn receptor in their complex compared to a control group not treated with the candidate substance as a substance for prevention or treatment of an NLRP3 inflammasome-associated disease.

In the present invention, the candidate substance, the first substance, the second substance, the cell, the interaction measurement method and the like are the same as the description of the method for screening a substance that regulates the interaction between GPCR19 and a P2Xn receptor in their complex, and the contents are quoted for the detailed description. Hereinafter, only the particular configuration of a method for screening a substance for prevention or treatment of NLRP3 inflammasome-associated disease will be described.

In the method according to the present invention, it may be judged that the interaction between GPCR19 and a P2Xn receptor in their complex is increased when any one or more of the following characteristics are exhibited as compared to a control group not treated with the candidate substance in step 4):
i) an increase in colocalization of GPCR19 and a P2Xn receptor in a cell surface, cytoplasm or nucleus;
ii) an increase in GPCR19-mediated signal transduction pathway activity, more specifically an increase in cAMP level or PKA activity;
iii) suppression of P2Xn receptor-mediated signal transduction pathway activity, more specifically a decrease in Ca⁺⁺ mobilization or inflammasomal activation, still more specifically a decrease in Ca⁺⁺ mobilization or a decrease in NLRP3, ASC, IL-1β, IL-18, caspase-1 or gasdermin D level, a decrease in NLRP3 inflammasome oligomerization, or a decrease in maturation of an IL-1β, IL-18 or caspase-1 immature form to a mature form; and
iv) a decrease in inflammatory cytokine level, more specifically a decrease in TNF-α, IL-1β, IL-18, RANTES or MCP-1 level.

In the method according to the present invention, the NLRP3 inflammasome-associated disease may be inflammatory diseases, degenerative diseases, metabolic diseases, neurological diseases or cancer, more specifically cancer, lupus, gout, sepsis, rheumatoid arthritis, osteoarthritis, juvenile idiopathic arthritis, ischemic retinopathy, age-related macular degeneration, chronic transplant rejection, psoriasis, psoriatic arthritis, atherosclerosis, atrial fibrillation, restenosis, obesity, pulmonary hypertension, chronic respiratory disease, cerebral infarction, angina pectoris, coronary artery disease, hypertension, stroke, anemia, migraine, nerve pain, arrhythmia, hemangioma, hyperlipidemia, peripheral vascular disease, vascular malformations, dementia, inflammatory bowel disease, osteoporosis, bone resorption, ulcerative colitis, respiratory distress syndrome, diabetes, non-alcoholic steatohepatitis (NASH), atopic dermatitis, actinic keratosis, delayed skin hypersensitivity disorder, Alzheimer's disease, Parkinson's disease, multiple sclerosis, multiple myeloma, asthma, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, epidermolytic ichthyosis, degenerative neuropathy, chronic obstructive pulmonary disease, pulmonary fibrosis, cryopyrin-associated periodic syndromes or endotoxin-induced diseases, but is not limited thereto.

In addition, the present invention also provides a method for preventing or treating an NLRP3 inflammasome-associated disease, which comprises administering to an individual a pharmaceutically effective amount of a substance that induces the interaction between GPCR19 and a P2Xn receptor in their complex.

In the present invention, the P2Xn receptor may be P2X1, P2X2, P2X3, P2X4, P2X5, P2X6 or P2X7 receptor, and may specifically be P2X7 receptor, but is not limited thereto.

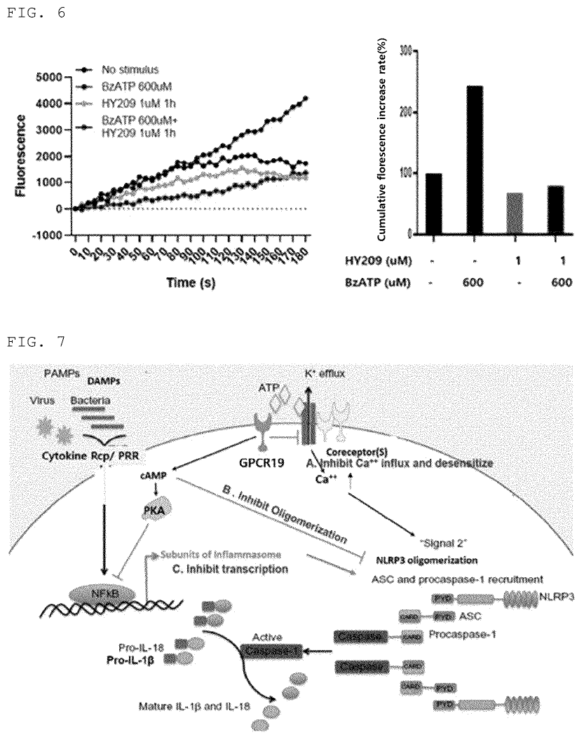

In the present invention, the substance that induces the interaction between GPCR19 and a P2Xn receptor in their complex induces the mutual binding between GPCR19 and a P2Xn receptor. As a result, GPCR19 and a P2Xn receptor form a complex, more specifically, a hetero-oligomeric complex to interact with each other. The interaction between GPCR19 and a P2Xn receptor in their complex is specifically that GPCR19 is activated to inhibit the activity of the P2Xn receptor, as a result, the GPCR19-mediated signal transduction pathway is activated and the P2Xn receptor-mediated signal transduction pathway is inactivated (see FIG. 6).

Accordingly, by administering a substance that induces the interaction between GPCR19 and a P2Xn receptor in their complex to an individual, any one or more of the following characteristics may be exhibited:
i) an increase in colocalization of GPCR19 and a P2Xn receptor in a cell surface, cytoplasm or nucleus;
ii) an increase in GPCR19-mediated signal transduction pathway activity, more specifically an increase in cAMP level or PKA activity;
iii) suppression of P2Xn receptor-mediated signal transduction pathway activity, more specifically a decrease in Ca⁺⁺ mobilization or inflammasomal activation, still more specifically a decrease in Ca⁺⁺ mobilization or a decrease in NLRP3, ASC, IL-1β, IL-18, caspase-1 or gasdermin D level, a decrease in NLRP3 inflammasome oligomerization, or a decrease in maturation of an IL-1β, IL-18 or caspase-1 immature form to a mature form; and
iv) a decrease in inflammatory cytokine level, more specifically a decrease in TNF-α, IL-1β, IL-18, RANTES or MCP-1 level.

In the present invention, the NLRP3 inflammasome-associated disease may be inflammatory diseases, degenerative diseases, metabolic diseases, neurological diseases or cancer, more specifically cancer, lupus, gout, sepsis, rheumatoid arthritis, osteoarthritis, juvenile idiopathic arthritis, ischemic retinopathy, age-related macular degeneration, chronic transplant rejection, psoriasis, psoriatic arthritis, atherosclerosis, atrial fibrillation, restenosis, obesity, pulmonary hypertension, chronic respiratory disease, cerebral infarction, angina pectoris, coronary artery disease, hypertension, stroke, anemia, migraine, nerve pain, arrhythmia, hemangioma, hyperlipidemia, peripheral vascular disease, vascular malformations, dementia, inflammatory bowel disease, osteoporosis, bone resorption, ulcerative colitis, respiratory distress syndrome, diabetes, non-alcoholic steatohepatitis (NASH), atopic dermatitis, actinic keratosis, delayed skin hypersensitivity disorder, Alzheimer's disease, Parkinson's disease, multiple sclerosis, multiple myeloma, asthma, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, epidermolytic ichthyosis, degenerative neuropathy, chronic obstructive pulmonary disease, pulmonary fibrosis, cryopyrin-associated periodic syndromes or endotoxin-induced diseases, but is not limited thereto.

The active ingredient according to the present invention, specifically, a substance that induces the interaction between GPCR19 and a P2Xn receptor in their complex is administered in a pharmaceutically effective amount. In the present invention, the “pharmaceutically effective amount” refers to an amount sufficient to treat a disease at a reasonable benefit/risk ratio applicable to medical treatment, and the effective dose level may be determined depending on factors including the kind of patient's disease, severity, drug activity, drug sensitivity, administration time, administration route and excretion rate, treatment period, and concomitant drugs and other factors well known in the medical arts. The composition of the present invention may be administered as a blended individual therapeutic agent or may be administered in combination with other therapeutic agents, may be administered sequentially or simultaneously with conventional therapeutic agents, and may be administered singly or multiple times. It is important to administer the composition in an amount, which is the minimum amount to obtain the maximum effect without side effects, while taking all of the factors into consideration, and this amount can be readily determined by those skilled in the art.

The active ingredient according to the present invention may include other ingredients as needed. Other ingredients include, but are not particularly limited to, pharmaceutical additives, such as stabilizers, surfactants, plasticizers, lubricants, solubilizers, buffering agents, sweeteners, substrates, adsorbents, seasoning agents, binders, suspending agents, antioxidants, brightening agents, coating agents, flavoring agents, perfumes, wetting agents, wetting regulators, defoamers, chewing agents, refreshing agents, coloring agents, dragees, isotonic agents, pH adjusters, softeners, emulsifiers, adhesives, adhesion enhancers, thickeners, thickening agents, foaming agents, excipients, dispersants, propellants, disintegrants, disintegration aids, fragrances, desiccants, antiseptics, preservatives, softening agents, solvents, dissolvents, dissolution aids, and glidants.

The active ingredient of the present invention may be administered to an individual, and the individual may be a mammal, specifically a human, a non-human mammal such as a non-human primate, an animal used in the model system (for example, a mouse and a rat used for screening, characterization and evaluation of pharmaceuticals), and other mammals, for example, an ape such as a rabbit, guinea pig, hamster, dog, cat, chimpanzee, gorilla, or monkey.

The active ingredient of the present invention may be administered orally or parenterally. In the case of being administered parenterally, the active ingredient may be administered by any one or more selected from the group consisting of transdermally, intravenously, intramuscularly, nasally and rectally, but is not limited thereto.

The administration form of the active ingredient according to the present invention is appropriately selected depending on the formulation method, the administration method, the patient's age, weight, disease, symptoms and the degree thereof, and the like, and is not particularly limited. Examples thereof include oral administration by tablets (including sublingual tablets and orally disintegrating tablets), granules, powders, liquids, syrup (including dry syrup), jellies, capsules (including soft capsules and microcapsules), and the like; and parenteral administration by injections (subcutaneous injections, intravenous injections, intramuscular injections, intraperitoneal injections and the like), vaginal tablets, vaginal ointments/creams, vaginal rings, vaginal gels or foams, vaginal inserts, suppositories (including rectal suppositories and vaginal suppositories), inhalants, transdermal absorbents, eye drops, nasal drops, and the like.

The dosage of the active ingredient according to the present invention is appropriately determined depending on the patient's age, sex, weight, disease, symptoms and the degree thereof.

In the formulation of the active ingredient according to the present invention, additives known in the art may be blended. For example, when a solid preparation for oral use is prepared, coated tablets, granules, powders, capsules and the like may be manufactured by adding an excipient, a binder, a disintegrant, a lubricant, a coloring agent, a flavoring agent, an odorant and the like to the active ingredient and then molding, granulating, encapsulating and the like the mixture by a conventional method. When a liquid preparation for oral use is prepared, an oral solution, a syrup and the like may be manufactured by adding a solvent such as purified water or ethanol, a dissolution aid, a suspending agent, an isotonic agent, a flavoring agent, a buffering agent, a stabilizer, an odorant and the like to the active ingredient and then distributing the crude solution by a conventional method. When an injection is prepared, subcutaneous, intramuscular, and intravenous injections and the like may be manufactured by adding a pH adjuster, a buffering agent, a stabilizer, an isotonic agent, a local anesthetic and the like to the active ingredient and then aseptically encapsulating the mixture in a container by a conventional method. When a rectal suppository is prepared, a preparation may be manufactured by adding an excipient, a surfactant and the like to the active ingredient and then mixing and molding the mixture by a conventional method.

As the dosage of the active ingredient according to the present invention, the active ingredient of the present invention may be administered at a dose of 0.0001 to 50 mg/kg or 0.001 to 50 mg/kg for adults when administered one time to several times a day in order to obtain a desirable effect. The dosage is not intended to limit the scope of the present invention in any way.

### EXAMPLES

Hereinafter, the present invention will be described in detail by way of Examples and Experimental Examples.

However, the following Examples and Experimental Examples only illustrate the present invention, and the contents of the present invention are not limited by the following Examples and Experimental Examples.

### <Example 1> Confirmation of Colocalization of GPCR19 and P2X7 Receptor

Colocalization of G-protein coupled receptor 19 (GPCR19) and P2X7 receptor was confirmed in various cells.

<1-1> Confirmation of Colocalization of GPCR19 and P2X7 Receptor in Keratinocyte

Colocalization of GPCR19 and P2X7 receptor in keratinocytes was confirmed.

Specifically, colocalization of GPCR19 and P2X7 receptor on the surface of HaCaT cells as keratinocytes was stimulated with DNCB (2,4-dinitrochlorobenzene)+TNF-α±sodium taurodeoxycholate (hereinafter referred to as ‘HY209’) and then analysis was performed using a confocal microscope. At this time, the cells were treated with DNCB to stimulate the NLRP3 inflammasome, and with TNF-α as an inflammatory inducer to stimulate the inflammatory response by activating the NF-κB signal. MFIs of P2X7 (green channel), GPCR19 (red channel) and colocalized P2X7/GPCR19 (yellow channel) were analyzed. MFIs in 4 to 5 ROIs (×600) were analyzed in each experimental set. In order to stain GPCR19 and P2X7 receptor, the HaCaT cells were treated with TNF-α (20 ng/ml)+DNCB (5 μg/ml)+HY209 (400 ng/ml) for 4 hours. The HaCaT cells were then aliquoted on a cover glass (Deckglaser, Luda-Konlgshofen, Germany), fixed with 4% paraformaldehyde for 10 minutes, and permeabilized with 0.3% Triton X-100 for 10 minutes. The HaCaT cells were blocked with PBS containing 1% BSA and 10% normal goat serum, stained with anti-P2X7 polyclonal Ab (Alomone labs, Jerusalem, Israel) or anti-GPCR19 polyclonal Ab (R & D, Systems Minneapolis, MN, USA) for 30 minutes at room temperature, and then stained with fluorochrome-labeled anti-HOST IgG. The cells after fluorescent antibody staining were mounted with a mounting medium containing DAPI (Vector laboratories, Burlingam, CA, USA), and observed using a confocal fluorescence microscope (Nikon, ECLIPSE Ti, New York, USA).

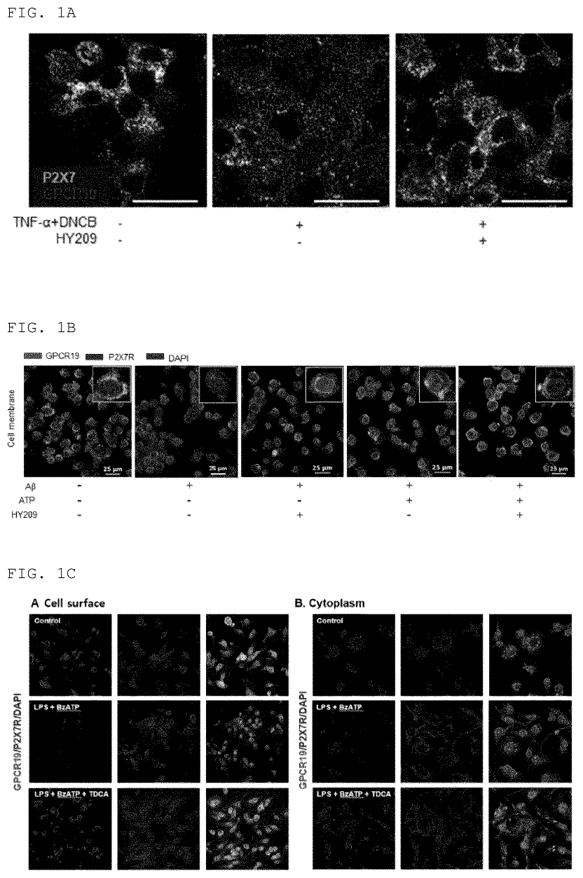

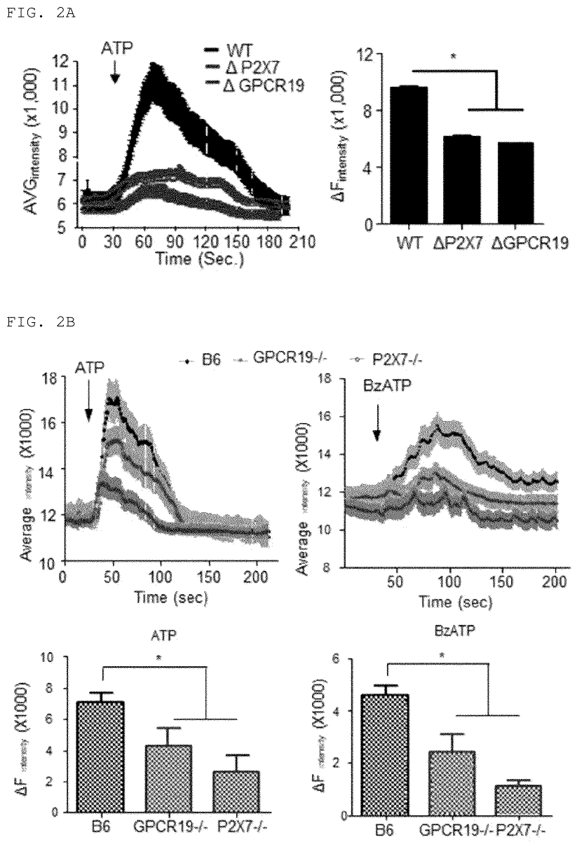

As a result, as illustrated in FIG. 1A, it has been confirmed that GPCR19 and P2X7 receptor colocalize on the HaCaT cell surface in the resting state, but the expression of P2X7 is significantly increased and the expression of GPCR19 is suppressed in the TNF-α+DNCB treatment group. However, in the group treated with HY209 together with TNF-α+DNCB, it has been confirmed that the expression of GPCR19 is increased, the expression of P2X7 is decreased, and GPCR19 and P2X7 receptor colocalize as in the resting state.

<1-2> Confirmation of Colocalization of GPCR19 and P2X7 Receptor in Microglia

Colocalization of GPCR19 and P2X7 receptor in microglia was confirmed.

Specifically, colocalization of GPCR19 and P2X7 receptor on the surface of BV2 cells as microglia was stimulated with Aβ±ATP±HY209 and then analysis was performed using a confocal microscope. At this time, the cells were treated with Aβ to stimulate the inflammatory response through the NF-κB signal in BV2 cells, and with ATP to activate P2X7 receptor. In order to stain GPCR19 and P2X7 receptor, BV2 cells were treated with Aβ (2 μM)+HY209 (400 ng/ml) for 1 hour. The BV2 cells were treated with ATP (1 mM) for an additional 1 hour prior to sample recovery. After treatment, the cells were stained in the same manner as in Example <1-1> so that cell surface GPCR19 was stained with anti-GPCR19 antibody and Alexa 488-labeled secondary antibody (green), P2X7R was immuno-stained with anti-P2X7R antibody and Alexa 446-labeled secondary antibody (red), and then nuclei were stained with DAPI, and mounting was performed. Thereafter, observation was performed using a confocal fluorescence microscope (Nikon, ECLIPSE Ti, New York, USA).

As a result, as illustrated in FIG. 1B, it has been confirmed that GPCR19 and P2X7 receptor colocalize on the BV2 cell surface in the resting state, but the expression of P2X7 is significantly increased and the expression of GPCR19 is suppressed in the Aβ single treatment group and the Aβ+ATP treatment group. However, in the Aβ+HY209 treatment group and the Aβ+ATP+HY209 treatment group, it has been confirmed that the expression of GPCR19 is increased and the expression of P2X7 is decreased. In particular, in the Aβ+ATP+HY209 treatment group, it has been confirmed that GPCR19 and P2X7 receptor colocalize as in the resting state.

<1-3> Confirmation of Colocalization of GPCR19 and P2X7 Receptor in Macrophage

Colocalization of GPCR19 and P2X7 receptor in macrophages was confirmed.

Specifically, colocalization of GPCR19 and P2X7 receptor on the surface and cytoplasm of BMDM (bone marrow-derived macrophage) cells as a macrophage was stimulated with LPS+BzATP±HY209, and then analysis was performed using a confocal microscope. At this time, the BMDM cells were treated with LPS as an inflammatory inducer to stimulate the inflammatory response by activating the NF-κB signal, and with BzATP to activate P2X7 receptor. In order to stain GPCR19 and P2X7 receptor, the BMDM cells were treated with LPS (10 ng/ml)±HY209 (400 ng/ml) for 1 hour. The BMDM cells were treated with BzATP (300 μm) for an additional 1 hour prior to sample recovery. After treatment, the cells were stained in the same manner as in Example <1-1> so that cell surface GPCR19 was stained with anti-GPCR19 antibody and Alexa 488-labeled secondary antibody (green), P2X7R was immuno-stained with anti-P2X7R antibody and Alexa 446-labeled secondary antibody (red), and then nuclei were stained with DAPI, and mounting was performed. Thereafter, observation was performed using a confocal fluorescence microscope (Nikon, ECLIPSE Ti, New York, USA).

As a result, as illustrated in FIG. 1C, it has been confirmed that GPCR19 and P2X7 receptor colocalize on the surface and cytoplasm of BMDM cells in the resting state, but the expression of P2X7 is significantly increased and the expression of GPCR19 is suppressed in the LPS+BzATP treatment group. However, in the group treated with HY209 together with LPS+BzATP, it has been confirmed that the expression of GPCR19 is increased, the expression of P2X7 is slightly decreased, and the GPCR19 and P2X7 receptor colocalize as in the resting state.

Through the results, it can be seen that GPCR19 and P2X7 receptor bind and interfere with each other inside and outside the cell. In addition, it can be seen that the expression of GPCR19 is decreased, the expression of P2X7 is increased, and in turn, the mutual binding action is not observed in the inflammation-induced situation, but the presence of HY209 can increase the expression level of GPCR19 and the mutual binding between GPCR19 and P2X7 receptor.

### <Example 2> Confirmation of Role of Mutual Binding Between GPCR19 and P2X7 Receptor in P2X7 Receptor-Mediated Ca⁺⁺ Mobilization

The role of mutual binding between GPCR19 and P2X7 receptor in Ca⁺⁺ mobilization by P2X7 receptor was confirmed.

<2-1> Confirmation of Ca⁺⁺ Mobilization in GPCR19 Knockout or P2X7 Knockout Macrophage

Ca⁺⁺ mobilization by P2X7 receptor in macrophages of GPCR19 gene knockout (KO) mice or P2X7 gene knockout mice was confirmed.

Specifically, BMDMs were obtained from three normal (wild-type, WT), GPCR19 KO or P2X7 KO mice. Then, the obtained BMDMs were treated with 20 μM ATP, attached to a glass coverslip, and then incubated in a physiological external solution consisting of NaCl 138 mM, KCl 5.6 mM, MgCl₂ 2 mM, HEPES 10 mM and pH 7.4 glucose 10 mM with 2 μM Fluo-4/AM in a 37° C. incubator for 30 minutes to observe intracellular Ca⁺⁺ mobilization. After that, the BMDMs were transferred to an open perfusion chamber to remove residual Fluo-4/AM, and the fluorescence level was measured using a fluorescence microscope (Nikon, Tokyo, Japan) under the conditions of excitation 494 nm and emission 506 nm. The microscope was equipped with an LED lamp (Andover, UK), an integrated shutter and a cooled EM-CCD camera, and the shutter and camera were controlled using MetaMorph software (Molecular Devices, US). In addition, a single cell was set as a region of interest (ROI), and a 16-bit grayscale image with 1×1 binning was taken with an exposure time of 1 second.

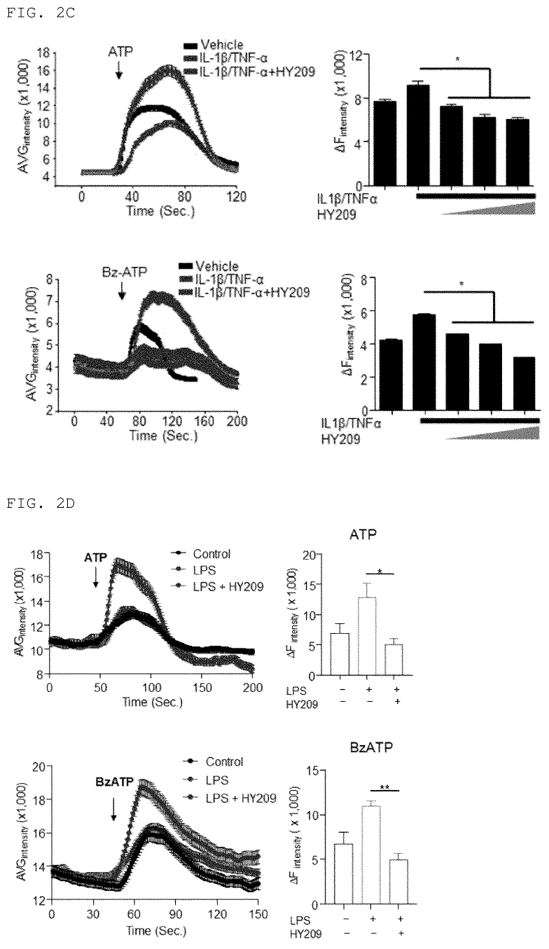

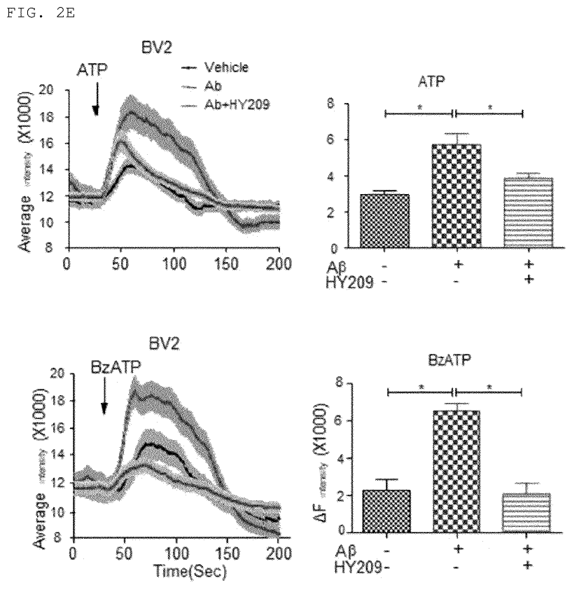

The fluorescence signal intensity measured using a fluorescence microscope was graphed. In the results, each bar in the graph denotes the SEM and the bold line denotes the average intensity (FIG. 2A, left panel). After three independent experiments, the sum of the measured data was presented (FIG. 2A, right panel).

As a result, as illustrated in FIG. 2A, it has been confirmed that intracellular Ca⁺⁺ mobilization increases when BMDMs of normal (WT) mice are stimulated with ATP. In contrast, intracellular Ca⁺⁺ mobilization is not significant when BMDMs of GPCR19 KO and P2X7 KO mice are stimulated with ATP. It has also been confirmed that there is a significant difference in intracellular Ca⁺⁺ mobilization between normal mice and GPCR19 KO and P2X7 KO mice.

<2-2> Confirmation of Ca⁺⁺ Mobilization in GPCR19 Knockout or P2X7 Knockout Microglia

Ca⁺⁺ mobilization by P2X7 receptor in microglia of GPCR19 KO mice or P2X7 KO mice was confirmed.

Specifically, microglia were obtained from six WT (B6), GPCR19 KO or P2X7 KO mice. Then, the obtained microglia were treated with ATP 40 μM (left) or BzATP 40 μM (right) and attached to a glass coverslip, and then intracellular Ca⁺⁺ mobilization was measured with Fura-2/AM using a fluorescence microscope in the same manner as in Example <2-1> and graphed. In the measured results, each bar in the graph denotes the SEM and the bold line denotes the average intensity (panels in first row). After three independent experiments, the sum of the measured data was presented (panels in second row).

As a result, as illustrated in FIG. 2B, it has been confirmed that intracellular Ca⁺⁺ mobilization increases when microglia of normal (WT) mice are stimulated with ATP or BzATP. In contrast, intracellular Ca⁺⁺ mobilization is not significant when BMDMs of GPCR19 KO and P2X7 KO mice are stimulated with ATP. It has also been confirmed that there is a significant difference in intracellular Ca⁺⁺ mobilization between normal mice and GPCR19 KO and P2X7 KO mice.

<2-3> Confirmation of Ca⁺⁺ Mobilization in Keratinocyte by Treatment with Inflammatory Inducer and HY209

Through <Example 1> described above, it has been confirmed that HY209 is an inducer of mutual binding between GPCR19 and P2X7 receptor. Accordingly, Ca⁺⁺ mobilization by P2X7 receptor in keratinocytes treated with inflammatory cytokines as an inflammatory inducer and with HY209 was confirmed.

Specifically, HaCaT cells were treated with IL-1β (10 ng/ml) and TNF-α (10 ng/ml), and after 4 hours, with HY209 (200, 400 and 800 ng/ml). Ca⁺⁺ mobilization measurement was started after treatment with HY209 (200 ng/ml), and the cells were treated with 20 μM of BzATP or ATP 50 seconds after the start of measurement. Intracellular Ca⁺⁺ mobilization was measured with Fura-2/AM using a fluorescence microscope in the same manner as in Example <2-1>, and graphed (FIG. 2C, left panels in first and second rows). After three independent experiments, the sum of the measured data was presented (FIG. 2C, right panels in first and second rows).

As a result, as illustrated in FIG. 2C, it has been confirmed that the inflammatory cytokines IL-1β and TNF-α increase Ca⁺⁺ mobilization by P2X7 receptor in HaCaT cells, but Ca⁺⁺ mobilization increased under the influence of IL-1β and TNF-α significantly decreases as the concentration of HY209 increases when the cells are treated with HY209 together with IL-1β and TNF-α.

<2-4> Confirmation of Ca⁺⁺ Mobilization in Macrophage by Treatment with Inflammatory Inducer and HY209

Ca⁺⁺ mobilization by P2X7 receptor in macrophages treated with LPS (lipopolysaccharide) as an inflammatory inducer and HY209 was confirmed.

Specifically, BMDM cells were treated with LPS (100 ng/ml)±HY209 (400 ng/ml) for 1 hour. After the treatment, Ca⁺⁺ mobilization measurement was started, and the cells were treated with ATP (20 μM) or BzATP (40 μM) in the presence of 2 mM CaCl₂) and 0.5 mM MgCl₂ 50 seconds after the start of measurement. Intracellular Ca⁺⁺ mobilization was measured with Fura-2/AM using a fluorescence microscope in the same manner as in Example <2-1>, and graphed (FIG. 2D, left panels in first and second rows). After three independent experiments, the sum of the measured data was presented (FIG. 2D, right panels in first and second rows).

As a result, as illustrated in FIG. 2D, it has been confirmed that LPS increases Ca⁺⁺ mobilization by P2X7 receptor in BMDM cells, but Ca⁺⁺ mobilization increased under the influence of LPS significantly decreases when the cells are treated with HY209 together with LPS.

<2-5> Confirmation of Ca⁺⁺ Mobilization in Microglia by Treatment with NLRP3 Inflammasome Activator and HY209

Ca⁺⁺ mobilization by P2X7 receptor in microglia treated with Aβ as an NLRP3 inflammasome activator and with HY209 was confirmed.

Specifically, BV2 cells were treated with Aβ (2 μM)±HY209 (400 ng/ml) for 1 hour. After the treatment, Ca⁺⁺ mobilization measurement was started, and the cells were treated with ATP (20 μM) or BzATP (40 μM) in the presence of 2 mM CaCl₂) and 0.5 mM MgCl₂ 50 seconds after the start of measurement. Intracellular Ca⁺⁺ mobilization was measured with Fura-2/AM using a fluorescence microscope in the same manner as in Example <2-1>, and graphed (FIG. 2E, left panels in first and second rows). After three independent experiments, the sum of the measured data was presented (FIG. 2E, right panels in first and second rows).

As a result, as illustrated in FIG. 2E, it has been confirmed that Aβ increases Ca⁺⁺ mobilization by P2X7 receptor in BMDM cells, but Ca⁺⁺ mobilization increased under the influence of Aβ significantly decreases when the cells are treated with HY209 together with Aβ.

Through the results, it has first been confirmed that calcium ion mobilization through P2X7 is dependent on the GPCR19 receptor. In addition, it can be seen that the accumulation of intracellular calcium ions is suppressed and inflammatory factors are reduced when calcium ion mobilization mediated by GPCR19 receptor is observed and mutual binding between GPCR19 and P2X7 receptor is induced using HY209 in the inflammation-induced situation.

### <Example 3> Confirmation of Role of Mutual Binding Between GPCR19 and P2X7 Receptor in Inflammation-Induced Cell

In order to investigate the role of mutual binding between GPCR19 and P2X7 receptor in inflammation-induced cells, changes in inflammasomal components and IL-1β expression in cells treated with DNCB/TNFα and HY209 were confirmed.

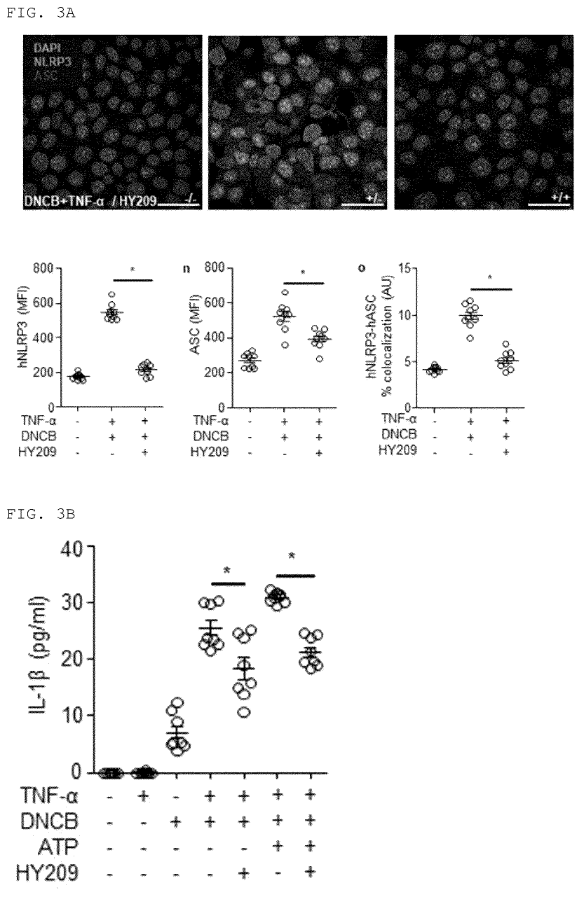

Specifically, HaCaT cells were treated with TNF-α (20 ng/ml)±HY209 (400 ng/ml) for 3 hours and then with DNCB (5 μg/ml) for additional 24 hours. For immunostaining, the cells were then aliquoted on a cover glass (Deckglaser, Luda-Konlgshofen, Germany), fixed with 4% paraformaldehyde for 10 minutes, permeabilized with 0.3% Triton X-100 for 10 minutes, and blocked with PBS containing 1% BSA and 10% normal goat serum for 1 hour. After that, in order to stain inflammasomal components, the cells were stained with anti-NLRP3 conjugated antibody (Abeam, Cambridge, UK) and anti-ASC Ab (Clone B-3, Santa Cruz Biotechnology, Inc. Dallas, Texas, USA) at 4° C. overnight, and then with Alexa Fluor 488-labeled or Alexa Fluor 532-labeled secondary conjugated antibody (Invitrogen, Carlsbad, CA, USA). The cells on the slide were mounted with a mounting medium containing DAPI (Vector laboratories, Burlingam, CA, USA) and observed using a confocal fluorescence microscope (Nikon, ECLIPSE Ti, New York, USA), and the results were graphed (FIG. 3A).

HaCaT cells were treated with DNCB (5 μg/ml)±TNF-α (20 ng/ml)±HY209 (400 ng/ml) for 24 hours and then with ATP for additional 3 hours, and the culture supernatant was recovered. Thereafter, the IL-1β concentration was measured according to the manufacturer's procedure using the IL-113 ELISA kit (R&D Systems Minneapolis, MN, USA) (FIG. 3B).

As a result, as illustrated in FIG. 3A, it has been confirmed that hNLRP3 and hASC expression and hNLRP3-ASC oligomerization are significantly increased in the TNF-α+DNCB treatment group. On the other hand, it has been confirmed that the increase in hNLRP3 and hASC expression and hNLRP3-ASC oligomerization is suppressed in the group treated with HY209 together with TNF-α+DNCB.

As illustrated in FIG. 3B, it has been confirmed that the production of IL-1β is significantly increased in the TNF-α+DNCB treatment group, but the increase in the production of IL-1β is suppressed in the group treated with HY209 together with TNF-α+DNCB. It has also been confirmed that the production of IL-1β is increased in the group treated with ATP together with TNF-α+DNCB compared to the TNF-α+DNCB treatment group, and the increase in the production of IL-1β is suppressed by HY209.

Through the results, it can be seen that when the mutual binding between GPCR19 and P2X7 receptor is inhibited, the cAMP-mediated NF-κB pathway is activated by the inactivation of GPCR19, the NLRP3 inflammasomal activation pathway is activated by the activation of P2X7 receptor, and in turn, the inflammatory response is promoted. On the other hand, it can be seen that a substance that induces mutual binding between GPCR19 and P2X7 receptor suppresses the inhibition of mutual binding between GPCR19 and P2X7 receptor to inactivate the pathways, and as a result, can alleviate the inflammatory response.

### <Example 4> Confirmation of Alleviation of Inflammatory Response in Inflammation-Induced Cell by Treatment with HY209

In order to investigate whether a substance that induces mutual binding between GPCR19 and P2X7 receptor alleviates the inflammatory response in inflammation-induced cells by suppressing the inhibition of mutual binding between GPCR19 and P2X7 receptor, cells treated with LPS and HY209 were additionally treated with ATP, and then changes in inflammasomal components and IL-113 expression were confirmed.

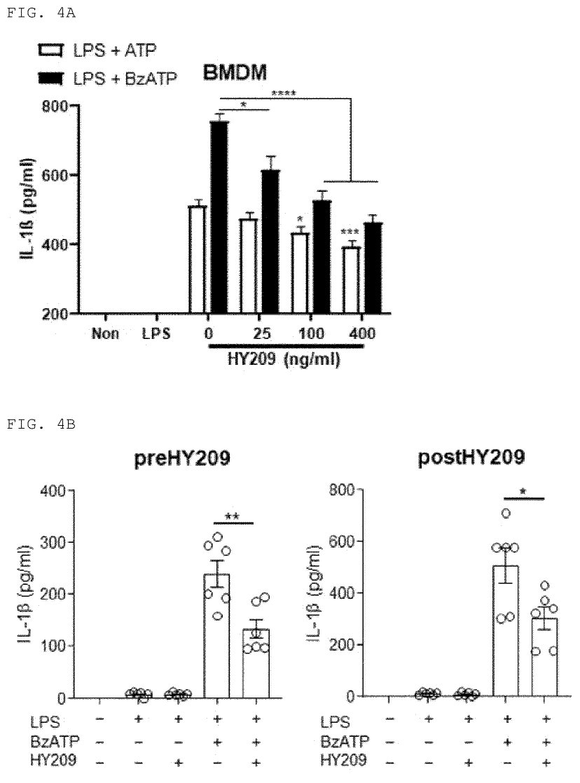

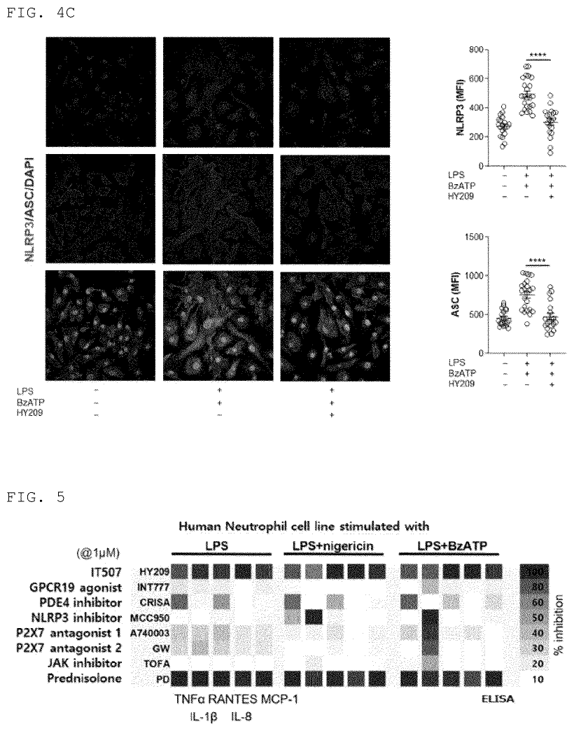

Specifically, in order to investigate the effect of treatment with HY209 at each concentration, BMDM cells were treated with LPS (10 ng/ml)±HY209 (0, 25, 100, 400 ng/ml) for 1 hour. The cells were treated with ATP (500 μm) or BzATP (300 μm) for an additional 1 hour before recovery of the culture supernatant of the sample. IL-1β concentration in the culture supernatant recovered was measured according to the manufacturer's procedure using the IL-1β ELISA kit (R & D Systems Minneapolis, MN, USA) (FIG. 4A).

In order to investigate the effect of pretreatment/posttreatment with HY209, BMDM cells were treated with HY209 (400 ng/ml) 1 hour before or 3 hours after treatment with LPS (10 ng/ml). Next, the cells were treated with BzATP (300 μm) for an additional 1 hour, and then the culture supernatant of the sample was recovered. IL-1β concentration in the culture supernatant recovered was measured according to the manufacturer's procedure using the IL-1β ELISA kit (R & D Systems Minneapolis, MN, USA) (FIG. 4B).

BMDM cells were treated with LPS (10 ng/ml)±HY209 (400 ng/ml) for 1 hour, and with BzATP (300 μm) for an additional 1 hour, inflammasomal components were stained in the same manner as in <Example 3>, and the cells were observed using a confocal fluorescence microscope (Nikon, ECLIPSE Ti, US) (FIG. 4C).

As a result, as illustrated in FIG. 4A, it has been confirmed that the increase in the production of IL-1β is suppressed in a HY209 concentration-dependent manner in both the group treated with HY209 together with LPS+ATP and the group treated with HY209 together with LPS+BzATP.

As illustrated in FIG. 4B, it has been confirmed that the increase in the production of IL-1β is suppressed by HY209 in both the group treated with HY209 before treated with LPS+ATP and the group treated with HY209 after treated with LPS+BzATP.

As illustrated in FIG. 4C, it has been confirmed that hNLRP3 and hASC expression and hNLRP3-ASC oligomerization are significantly increased in the LPS+BzATP treatment group. On the other hand, in the group treated with HY209 together with LPS+BzATP, it has been confirmed that the increase in hNLRP3 and hASC expression and hNLRP3-ASC oligomerization is suppressed.

Through the results, however, it can be seen that a substance that induces mutual binding between GPCR19 and P2X7 receptor suppresses the inhibition of mutual binding between GPCR19 and P2X7 receptor and induces mutual binding between GPCR19 and P2X7 receptor to inactivate the cAMP-mediated NF-κB pathway and NLRP3 inflammasomal activation pathway, and as a result, can prevent and alleviate the inflammatory response.

### <Example 5> Cytokine Measurement Using Cytometric Bead Array (CBA)

The inflammatory response alleviating effect of a substance that induces mutual binding between GPCR19 and P2X7 receptor and that of a known inflammasome inhibitor in inflammation-induced cells were analyzed and compared.

Specifically, cocktail beads were prepared by mixing fluorescent beads bound with antibodies to five types of inflammatory cytokines, TNF-α, RANTES, MCP-1, IL-1β and IL-8 to be confirmed. Next, 50 μl of the prepared cocktail beads, the prepared sample, and the standard sample were reacted at room temperature for 1 hour in a dark environment. At this time, as the sample, 1 μM of HY209 confirmed in Examples described above was used as a substance inducing mutual binding between GPCR19 and P2X7 receptor, 1 μM of INT777 (Cat. No. HY-15677, MedChemExpress) was used as a GPCR19 agonist, 1 μM of crisaborole (Eucrisa, Pfizer) was used as a PDE4 inhibitor, 1 μM of MCC950 (CAS No. 256373-96-3, Calbiochem) was used as a NLRP3 inhibitor, 1 μM of A740003 (CAS No. 861393-28-4, Sigma-Aldrich) and 1 μM of GW791343 (CAS No. 309712-55-8, Tocris) were used as P2X7 antagonists, and 1 μM of tofacitinib (CAS No. 540737-29-9, Sigma-Aldrich) was used as a JAK inhibitor. As a positive control group, 1 μM of prednisolone, a corticosteroid was used. The reaction mixture was transferred to a new tube by the required amount so that the phycoerythrin (PE) detection reagent was 1 μl/sample, and washed with the washing buffer, the capture bead diluent was added so as to be contained by 50 μl per each sample through the calculation of volume, and the reaction was conducted at room temperature for 15 minutes in a dark environment. Next, 50 μl of the prepared PE detection reagent was added into the tube in which the cocktail beads, the prepared sample, and the standard sample were reacting, mixing was thoroughly performed, and then the reaction was further conducted for 2 hours. After all reactions were completed, 1 ml of washing buffer was added, centrifugation was performed at 200 g for 5 minutes, then the supernatant was removed, 300 μl of washing buffer was added, and then quantitative measurement of the 5 types of inflammatory cytokines was performed using a flow cytometer (CANTO II FACS, BD Biosciences, US).

As a result, as illustrated in FIG. 5, it has been confirmed that the inhibitory effect of HY207 on five types of inflammatory cytokines produced by the inflammasome stimulation signal is superior.

### <Example 6> Confirmation of Regulation of Ion Channel Using Potassium Ion Channel Assay

Changes in ion channel activity in inflammation-induced cells by HY209, a GPCR19 agonist were analyzed and compared.

Specifically, U937 cells were used to investigate whether the activity of ion channel was changed by the regulation of GPCR19. U937 cells were differentiated by treatment with PMA (25 nM). After that, a fluorescent probe was added and the reaction was conducted for 1 hour. Thereafter, the cells were treated with BzATP (600 μM), and the cumulative fluorescence value was measured using an ELISA instrument to investigate whether the ion channel was activated. As a result of Example, the fact that the ion channel is activated when U937 cells differentiated by PMA are treated with BzATP (600 μM) has been confirmed by an increase in cumulative fluorescence. At this time, it has been confirmed that the cumulative fluorescence value increased by BzATP is decreased by 100% when the cells are treated with HY209 (1 μM) 1 hour before the treatment with BzATP.

Through the results, it has been confirmed that HY209 regulates GPCR19 and effectively regulates P2X7, an important ion channel in the inflammatory response.

Through the results of <Example 1> to <Example 6>, as illustrated in the schematic diagram of FIG. 7, a physiological mechanism has been confirmed in which the NLRP3 inflammasomal activation pathway mediated by P2X7 is regulated by GPCR19 in the DAMP stress inflammation-induced situation due to biomaterials such as PAMP and ATP caused by microorganisms. In addition, it has been confirmed that the inflammatory response initiated from P2X7 can be prevented or alleviated when mutual binding between GPCR19 and P2X7 receptor is induced during an inflammatory response by utilizing a substance that induces mutual binding between GPCR19 and P2X7 receptor. In addition, it has been confirmed that HY209 exhibits a superior effect of preventing or alleviating the inflammatory response by inducing mutual binding between GPCR19 and P2X7 receptor unlike conventional inflammasome inhibitors and GPCR19 agonists.

### INDUSTRIAL APPLICABILITY

According to a GPCR19-P2Xn receptor complex and its use, it is possible to screen substances and prevent or treat NLRP3 inflammasome-associated diseases, and thus the GPCR19-P2Xn receptor complex can be usefully utilized in medicine and pharmaceutical fields and the like.

1. A method for screening a substance that regulates interaction between GPCR19 and a P2Xn receptor in their complex, the method comprising:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance having a difference in interaction between GPCR19 and a P2Xn receptor in their complex compared to a control group not treated with the candidate substance as a substance that regulates interaction between GPCR19 and a P2Xn receptor in their complex.

2. The method according to claim 1,
wherein the first substance is an inflammatory inducer or an NLRP3 inflammasome activator.

3. The method according to claim 2,
wherein the inflammatory inducer is a TLR (Toll-like receptor) ligand or a cytokine, and preferably is selected from the group consisting of LPS (lipopolysaccharide), peptidoglycan, TNF-α, IL-1β and IL-17.

4. The method according to claim 2,
wherein the inflammatory inducer induces inflammation through NF-κB signal transduction pathway.

5. (canceled)

6. The method according to claim 2,
wherein the NLRP3 inflammasome activator is amyloid-β (Aβ).

7. The method according to claim 1,
wherein the second substance is a P2Xn receptor agonist, and preferably ATP, BzATP or nigericin.

8. (canceled)

9. The method according to claim 1,
wherein the P2Xn receptor is selected from the group consisting of P2X1, P2X2, P2X3, P2X4, P2X5, P2X6 and P2X7 receptors.

10. The method according to claim 1,
wherein the cell heterologously or endogenously expresses GPCR19 and a P2Xn receptor.

11. The method according to claim 1,
wherein the cell is selected from the group consisting of stem cells, animal cells, insect cells and plant cells.

12. The method according to claim 1,
wherein the cell is selected from the group consisting of myeloid cells, lymphoid cells, microglia, macrophages, bone marrow-derived macrophages, neutrophils, monocytes, epithelial cells, dermal cells, endothelial cells, myocytes, germ cells, skin cells, immune cells and cancer cells.

13. The method according to claim 1,
wherein the interaction between GPCR19 and a P2Xn receptor in their complex in step 3) is measured by analyzing any one or more of the following characteristics:
i) a change in colocalization of GPCR19 and a P2Xn receptor in a cell surface, cytoplasm or nucleus;
ii) a change in GPCR19-mediated signal transduction pathway activity, and wherein the change in GPCR19-mediated signal transduction pathway activity is a change in cAMP level or PKA activity;
iii) a change in P2Xn receptor-mediated signal transduction pathway activity, and wherein the change in P2Xn receptor-mediated signal transduction pathway activity is a change in Ca⁺⁺ mobilization or inflammasomal activation; and
iv) a change in inflammatory cytokine level, and wherein the inflammatory cytokine is selected from the group consisting of TNF-α, IL-1β, IL-18, RANTES and MCP-1.

14-15. (canceled)

16. The method according to claim 13,
wherein the change in inflammasomal activation of the characteristic iii) is a change in NLRP3, ASC, pro-IL-1β, IL-1β, pro-IL-18, IL-18, pro-caspase-1, caspase-1 or gasdermin D level, a change in NLRP3 inflammasome oligomerization, or a change in maturation of an IL-1β, IL-18 or caspase-1 immature form to a mature form.

17. (canceled)

18. A method for screening a substance for prevention or treatment of an NLRP3 inflammasome-associated disease, the method comprising:
1) treating a cell expressing GPCR19 and a P2Xn receptor with a candidate substance and a first substance;
2) treating the cell of step 1) with a second substance;
3) measuring interaction between GPCR19 and a P2Xn receptor in their complex in the cell of step 2); and
4) as a result of the interaction measurement in step 3), judging a candidate substance that increases interaction between GPCR19 and a P2Xn receptor in their complex compared to a control group not treated with the candidate substance as a substance for prevention or treatment of an NLRP3 inflammasome-associated disease.

19. The method according to claim 18,
wherein the first substance is an inflammatory inducer or an NLRP3 inflammasome activator.

20. The method according to claim 18,
wherein the second substance is a P2Xn receptor agonist.

21. The method according to claim 18,
wherein it is judged that interaction between GPCR19 and a P2Xn receptor in their complex is increased when any one or more of the following characteristics are exhibited compared to a control group not treated with the candidate substance in step 4):
i) an increase in colocalization of GPCR19 and a P2Xn receptor in a cell surface, cytoplasm or nucleus;
ii) an increase in GPCR19-mediated signal transduction pathway activity, and preferably an increase in cAMP level or PKA activity;
iii) suppression of P2Xn receptor-mediated signal transduction pathway activity, and preferably a decrease in Ca⁺⁺ mobilization or inflammasomal activation; and
iv) a decrease in inflammatory cytokine level, and wherein the inflammatory cytokine is selected from the group consisting of TNF-α, IL-1β, IL-18, RANTES and MCP-1.

22-23. (canceled)

24. The method according to claim 21,
wherein the decrease in inflammasomal activation of the characteristic iii) is a decrease in NLRP3, ASC, IL-1β, IL-18, caspase-1 or gasdermin D level, a decrease in NLRP3 inflammasome oligomerization, or a decrease in maturation of an IL-1β, IL-18 or caspase-1 immature form to a mature form.

25. (canceled)

26. The method according to claim 18,
wherein the NLRP3 inflammasome-associated disease is selected from the group consisting of inflammatory diseases, degenerative diseases, metabolic diseases, neurological diseases and cancer, and preferably is selected from the group consisting of cancer, lupus, gout, sepsis, rheumatoid arthritis, osteoarthritis, juvenile idiopathic arthritis, ischemic retinopathy, age-related macular degeneration, chronic transplant rejection, psoriasis, psoriatic arthritis, atherosclerosis, atrial fibrillation, restenosis, obesity, pulmonary hypertension, chronic respiratory disease, cerebral infarction, angina pectoris, coronary artery disease, hypertension, stroke, anemia, migraine, nerve pain, arrhythmia, hemangioma, hyperlipidemia, peripheral vascular disease, vascular malformations, dementia, inflammatory bowel disease, osteoporosis, bone resorption, ulcerative colitis, respiratory distress syndrome, diabetes, non-alcoholic steatohepatitis (NASH), atopic dermatitis, actinic keratosis, delayed skin hypersensitivity disorder, Alzheimer's disease, Parkinson's disease, multiple sclerosis, multiple myeloma, asthma, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, epidermolytic ichthyosis, degenerative neuropathy, chronic obstructive pulmonary disease, pulmonary fibrosis, cryopyrin-associated periodic syndromes and endotoxin-induced diseases.

27. (canceled)

28. An isolated GPCR19-P2Xn receptor complex, which is for screening a substance for prevention or treatment of an NLRP3 inflammasome-associated disease.

29. A method for preventing or treating an NLRP3 inflammasome-associated disease, the method comprising administering to an individual a pharmaceutically effective amount of a substance that induces interaction between GPCR19 and a P2Xn receptor in their complex.

30. The method according to claim 29,
wherein the substance that induces interaction between GPCR19 and a P2Xn receptor in their complex exhibits any one or more of the following characteristics when administered to an individual:
i) an increase in colocalization of GPCR19 and a P2Xn receptor in a cell surface, cytoplasm or nucleus;
ii) an increase in GPCR19-mediated signal transduction pathway activity, and preferably an increase in cAMP level or PKA activity;
iii) suppression of P2Xn receptor-mediated signal transduction pathway activity, and preferably a decrease in Ca⁺⁺ mobilization or inflammasomal activation; and
iv) a decrease in inflammatory cytokine level, and wherein the inflammatory cytokine is selected from the group consisting of TNF-α, IL-1β, IL-18, RANTES and MCP-1.

31-32. (canceled)

33. The method according to claim 30,
wherein the decrease in inflammasomal activation of the characteristic iii) is a decrease in NLRP3, ASC, IL-1β, IL-18, caspase-1 or gasdermin D level, a decrease in NLRP3 inflammasome oligomerization, or a decrease in maturation of an IL-1β, IL-18 or caspase-1 immature form to a mature form.

34. (canceled)

35. The method according to claim 30,
wherein the NLRP3 inflammasome-associated disease is selected from the group consisting of inflammatory diseases, degenerative diseases, metabolic diseases, neurological diseases and cancer, and preferably is selected from the group consisting of cancer, lupus, gout, sepsis, rheumatoid arthritis, osteoarthritis, juvenile idiopathic arthritis, ischemic retinopathy, age-related macular degeneration, chronic transplant rejection, psoriasis, psoriatic arthritis, atherosclerosis, atrial fibrillation, restenosis, obesity, pulmonary hypertension, chronic respiratory disease, cerebral infarction, angina pectoris, coronary artery disease, hypertension, stroke, anemia, migraine, nerve pain, arrhythmia, hemangioma, hyperlipidemia, peripheral vascular disease, vascular malformations, dementia, inflammatory bowel disease, osteoporosis, bone resorption, ulcerative colitis, respiratory distress syndrome, diabetes, non-alcoholic steatohepatitis (NASH), atopic dermatitis, actinic keratosis, delayed skin hypersensitivity disorder, Alzheimer's disease, Parkinson's disease, multiple sclerosis, multiple myeloma, asthma, rhinitis, hepatitis, keratitis, gastritis, enteritis, nephritis, bronchitis, pleurisy, peritonitis, spondylitis, pancreatitis, inflammatory pain, urethritis, cystitis, burn inflammation, dermatitis, periodontitis, gingivitis, epidermolytic ichthyosis, degenerative neuropathy, chronic obstructive pulmonary disease, pulmonary fibrosis, cryopyrin-associated periodic syndromes and endotoxin-induced diseases.

36. (canceled)
